(19) United States
(12) Patent Application Publication
(10) Pub. No.: US 2014/0106342 A1
(43) Pub. Date: Apr. 17, 2014

(54) METHOD OF ASSAYING DNA TOPOISOMERASES AND DNA BINDING PROTEINS USING HIGH THROUGHPUT SCREENING

(71) Applicants: Mark T. Muller, Port Orange, FL (US); Vidusha Cyril, Orlando, FL (US)

(72) Inventors: Mark T. Muller, Port Orange, FL (US); Vidusha Cyril, Orlando, FL (US)

(21) Appl. No.: 13/984,948

(22) PCT Filed: Feb. 10, 2012
(86) PCT No.: PCT/US12/24758

§ 371 Date: Dec. 26, 2013

Related U.S. Application Data
(60) Provisional application No. 61/441,377, filed on Feb. 10, 2011.

(51) Int. Cl. C12Q 1/533

(52) U.S. Cl. CPC C12Q 1/533
USPC 435/6.1; 435/174

(57) ABSTRACT

A novel high throughput screening (HTS) technique to rapidly identify eukaryotic topoisomerase I active agents is presented. The method is based on genetic tagging of the topoisomerase I enzyme to directly immobilize the enzyme on a solid surface in a microtiter well format. For HTS operations, DNA is added to the wells and a fraction of the input plasmid is retained on the enzyme attached to the solid phase substratum. The retained DNA is detected by Picogreen fluorescence. Compounds that result in an increase in Picogreen staining represent potential topoisomerase interfacial poisons while those that reduce fluorescence report the presence of a catalytic inhibitor; therefore, the solid phase assay represents a ‘bimodal’ readout that reveals mechanisms of action. In addition to specific topoisomerase targeting drugs, the method also weakly detects other relevant anticancer agents, such as potent DNA alkylating and intercalating compounds; therefore, topoisomerase I HTS represents an excellent tool for searching and identifying novel genotoxic agents. This solid phase HTS is rapid, robust, economical and scalable for large library screens.

### CROSS REFERENCE TO RELATED APPLICATIONS

This application is related to U.S. Provisional Application No. 61/441,377 filed Feb. 10, 2011 to which priority is claimed under 35 USC 119, and whose description is incorporated herein in its entirety.

### FIELD OF INVENTION

This invention relates to assays. Specifically this invention is related to the detection of novel agents that target DNA binding proteins by assaying for DNA topoisomerases using high throughput screening.

### BACKGROUND OF THE INVENTION

The double helical structure of the DNA imposes topological constraints when the duplex is read as a template. Thus, during processes such as replication or transcription, strand separation generates alterations in twist which causes the strands to writhe up or downstream of the site of polymerization. These structural changes can impede reading of the template and inhibit the central genetic process. (Kanaar R, Cozzarelli N R: Roles of supercoiled DNA structure in DNA transactions. Current Opinion in Structural Biology 1992 2:369-379; Wang J C: DNA Topoisomerases. Annual Review of Biochemistry 1996 65:635-692). Torsional stress is known to be regulated by a group of ubiquitous nuclear enzymes known as DNA topoisomerases. (Leppard J B, Champoux J J: Human DNA topoisomerase I: relaxation, roles, and damage control. Chromosoma 2005 114:75-85; Wang J C: Cellular roles of DNA topoisomerases: a molecular perspective. Nat Rev Mol Cell Biol 2002 3:430-440)

The topological changes are based on alterations in DNA linking number through concerted breakage and rejoining of one (type I enzymes) or both (type II enzyme) DNA sugar phosphate backbones. (Champoux J J: DNA TOPOISOMERASES: Structure, Function, and Mechanism. Annual Review of Biochemistry 2001 70:369; Corbett K D, Berger J M: STRUCTURE, MOLECULAR MECHANISMS, AND EVOLUTIONARY RELATIONSHIPS IN DNA TOPOISOMERASES. Annual Review of Biophysics & Biomolecular Structure 2004 33:95-C-96; Forterre P, Gribaldo S, Gadelle D, Serre M-C: Origin and evolution of DNA topoisomerases. Biochimie 2007 89:427-446; Leppard J B, Champoux J J: Human DNA topoisomerase I: relaxation, roles, and damage control. Chromosoma 2005 114:75-85; McClendon A K, Osheroff N: DNA topoisomerase II, genotoxicity, and cancer. Mutation Research/Fundamental and Molecular Mechanisms of Mutagenesis 2007 623:83-97; Wang J C: Cellular roles of DNA topoisomerases: a molecular perspective. Nat Rev Mol Cell Biol 2002; 3:430-440; Wang J C: DNA Topoisomerases. Annual Review of Biochemistry 1996; 65:635-692)

The reaction mechanism is tightly coupled as a transesterification event such that the broken DNA intermediate is transitory and does not accumulate during the normal course of a topological adjustment (defined as a change in DNA linking number). Inappropriate cleavage events are a danger to somatic cell survival and thus, the topoisomerase/DNA cleavage intermediate is not detected in normal cells. (Champoux J J: DNA TOPOISOMERASES: Structure, Function, and Mechanism. Annual Review of Biochemistry 2001 70:369; Stewart L, Redinbo M R, Qiu X, Hol W G J, Champoux J J: A Model for the Mechanism of Human Topoisomerase I. Science 1998; 279:1534-1541)

Importantly, compounds that disrupt the equilibrium between cleaved and uncleaved DNA in the topoisomerase reaction cycle are excellent anti-cancer agents. (McClendon A K, Osheroff N: DNA topoisomerase II, genotoxicity, and cancer. Mutation Research/Fundamental and Molecular Mechanisms of Mutagenesis 2007 623:83-97; Pommier Y: Topoisomerase I inhibitors: camptothecins (CPT) and beyond. Nat Rev Cancer 2006; 6:789-802)

The clinically approved drugs are known as interfacial poisons (IFP) since they stabilize the cleavage intermediate and fragment the genome, usually during S-phase, thereby eliminating the tumor cell with some degree of selectivity over normal resting cells. (Marchand C, Antony S, Kohn K W, Cushman M, Ioanoviciu A, Staker B L, Burgin A B, Stewart L, Pommier Y: A novel norindenoisoquinoline structure reveals a common interfacial inhibitor paradigm for ternary trapping of the topoisomerase I-DNA covalent complex. Molecular Cancer Therapeutics 2006 5:287-295; McClendon A K, Osheroff N: DNA topoisomerase II, genotoxicity, and cancer. Mutation Research/Fundamental and Molecular Mechanisms of Mutagenesis 2007 623:83-97).

A less explored aspect of drug action involves agents that inhibit topoisomerase enzymatic function and these are termed catalytic inhibitor compounds (CIC). The CIC may affect either protein or DNA structure rendering the topoisomerase unable to engage the cycle of breakage/rejoining. Such agents may be less specific but are nonetheless potentially important, especially given the importance of topoisomerase (topo) in many central genetic events.

In the case of topo I, there are a large number of interfacial poisons, including camptothecins (and congeners), indolcarbazoles, NSC314622, indenoisoquinolines, among others. (Hsiang Y H, Hertzberg R, Hecht S, Liu L F: Camptothecin induces protein-linked DNA breaks via mammalian DNA topoisomerase I. Journal of Biological Chemistry 1985 260:14873-14878; Staker B L, Hjerrild K, Feese M D, Behnke C A, Burgin A B, Stewart L: The mechanism of topoisomerase I poisoning by a camptothecin analog. Proc Natl Acad Sci USA 2002 99:15387-15392). Camptothecin derivatives are particularly prevalent in this group. There are far fewer topo I CIC in general. (Bendetz-Nezer S, Gazit A, Priel E: DNA Topoisomerase I As One of the Cellular Targets of Certain Tyrphostin Derivatives. Molecular Pharmacology 2004 66:627-634; Chen A Y, Liu L F: DNA Topoisomerases: Essential Enzymes and Lethal Targets. Annual Review of Pharmacology and Toxicology 1994 34:191-218; Malina J, Vrana O, Brabec V: Mechanistic studies of the modulation of cleavage activity of topoisomerase I by DNA adducts of mono- and bi-functional PtII complexes. Nucleic Acids Research 2009 37:5432-5442)

Over the years, a number of programs have focused on searching for novel topo I targeting agents in order to improve, on the existing panoply of camptothecin congeners and find completely novel drug archetypes that can overcome the negative aspects of topo I mediated therapeutics (such as stability, collateral tissue damage, multiple drug resistance).

### SUMMARY OF THE INVENTION

It has now been realized that there is a pressing need for a high-throughput screening (HTS) platform that is mechanistically sophisticated such that novel compounds can be readily identified. Ideally, the readout should also provide clues as to the nature of the drug action on topo I (IFP versus CIC).

While some methods exist that can used (i.e., agarose gel electrophoresis, SDS-K+, ICE Bioassay) such methods are not readily HTS adaptable and are not easily scalable. (Muller M T: Quantitation of eukaryotic topoisomerase I reactivity with DNA. Preferential cleavage of supercoiled DNA. Biochimica et Biophysica Acta (BBA)—Gene Structure and Expression 1985 824:263-267; Subramanian D, Furbee C S, Muller M T: ICE Bioassay. DNA Topoisomerase Protocols: Volume II: Enzymology and Drugs, 2000: 137-147; Trask D, DiDonato J, Muller M: Rapid Detection and isolation of covalent DNA/protein complexes: application to topoisomeraes I and II. EMBO Journal 1984 3:671-676; Trask D K, Muller M T: Biochemical characterization of topoisomerase I purified from avian erythrocytes. Nucleic Acids Research 1983 11:2779-2800; Trask D K, Muller M T: Stabilization of type I topoisomerase-DNA covalent complexes by actinomycin D. Proceedings of the National Academy of Sciences of the United States of America 1988 85:1417-1421)

Several other new assays have been proposed: to improve our ability to find new topo active agents (triplex based assays, dual color fluorescence spectroscopy, and Surface Plasmon Resonance); however, such methods are a bit complex and technologically intense. (Shapiro A, Jahic H, Prasad S, Ehmann D, Thresher J, Gao N, Hajec L: A Homogeneous, High-Throughput Fluorescence Anisotropy-Based DNA Supercoiling Assay. Journal of Biomolecular Screening; 15:1088-1098; Maxwell A, Burton N P, O'Hagan N: High-throughput assays for DNA gyrase and other topoisomerases. Nucleic Acids Research; 34:e104-e104; Tsai H-P, Lin L-W, Lai Z-Y, Wu J-Y, Chen C-E, Hwang J, Chen C-S, Lin C-M: Immobilizing topoisomerase I on a surface plasmon resonance biosensor chip to screen for inhibitors. Journal of Biomedical Science; 17:49). Most of the assays in this area have focused on analysis of the products of the reaction or changes in DNA topology which makes them derivative methods. See, for example, U.S. Pat. No. 6,197,527 to Lynch et al. However, such assays only detect IFPs, not CICs. The present invention does not require the use of antibodies; measures the functional activity of topo in a solid phase format; and provides a mechanistic readout of both IFPs and CICs.

Reading the DNA template during transcription and replication creates topological alterations in the helix that must be adjusted through the concerted activity of DNA topoisomerases. These are ubiquitous enzymes and with a few exceptions function in similar ways in pro and eukaryotic systems. In eukaryotes, topoisomerases are attractive anti-cancer drug targets due to their ability to damage the cancer cell genome in the presence of drugs that abort the normal cycle of breakage/reunion of the DNA backbone.

Two major subdivisions of topoisomerases are the type I enzymes, which make single strand transient nicks and the type II enzymes which break/reseal both strands. Clinically approved anti-cancer agents are usually highly specific with many more type II topoisomerase drugs known compared to the type I class. Typically, drug discovery involves mechanism based assays using agarose gels which are not amenable to high-throughput screening (HTS) operations. Disclosed herein is the development and testing of a novel HTS technique to address this need.

In regard to one illustrative embodiment example, a method is disclosed that is based on immobilizing the enzyme on a solid surface in a microtiter well format under conditions that retain catalytic activity. For HTS operations, DNA is added to the wells and a fraction of the input plasmid is retained on the enzyme that is attached to the solid phase substratum. The retained DNA is detected by ultra-sensitive fluorescence. Compounds that result in an increase in enhanced fluorescence represent potential topoisomerase interfacial poisons while those that reduce fluorescence indicate presence of a possible catalytic inhibitor; therefore, the solid phase assay represents a ‘bimodal’ readout. The method has been demonstrated to work with known interfacial poisons and is responsive to conditions that push the enzyme into a distributive mode, such as catalytic type inhibitors. This solid phase HTS is rapid, robust, economical and scalable for larger library screens.

Embodiments of the invention can detect both topo I inhibitors and poisons; thus, this novel assay is a bimodal metric that classifies potential ‘hits’ as being inhibitory (thereby blocking enzyme action on DNA) or an interfacial poison (traps the cleavage intermediate). This is a powerful mechanistic screen that gives useful information on potential leads.

Further embodiments are described below.

### BRIEF DESCRIPTION OF THE DRAWINGS

For a fuller understanding of the invention, reference should be made to the following detailed description, taken in connection with the accompanying drawings, in which:

FIG. 1 is an image depicting the purification of human Topo I as a His-tag product. Baculovirus infected SF-9 cells were harvested as described in “Materials and Methods” and the crude extract loaded onto the nickel column. Input, FT (flow through, column void), wash fractions are indicated. The imidazole eluted fractions (E1-E6) are marked. The E2 fraction contained about 2.24 mg of topo I, corresponding to about 22 uM yield with over 2×10⁶ units of enzyme. This fraction was diluted from about 0 to about 1:1500 and assayed for relaxation of plasmid DNA (upper gel image). Cleavage activity of His-tag topo I was assessed in the lower gel. Purified topo I (about 500 units) was incubated with about zero or about 1 uM CPT and about 100 ng of pHOT1 supercoiled DNA for about 30 min at about 37° C. and reactions terminated with about 1% SDS. After proteinase K digestion, samples were loaded directly onto a 1% agarose gel containing about 0.5 ug/mL ethidium bromide. The positions of supercoiled (form I), relaxed (form I_{R}) and nicked open circular (form II) DNAs are indicated. Low levels of topo I cleavage products are visible in the minus drug control.

FIG. 2 is a series of images depicting Topo I activity on affinity beads. (A) Nickel sepharose beads were incubated with purified His-tag topo I or BSA. Input proteins and FT (flow through) fractions are marked. The beads were washed by centrifugation (about 10 bead volumes per wash) and each wash fraction collected and loaded onto an SDS-PAGE for analysis by staining with coomassie blue. Below the input lane is a topo I relaxation assay of affinity beads that were washed to remove unbound enzyme. Lanes 1 and 2 contain a supercoiled DNA reference marker and topo I control, respectively. Lanes 3 and 4 are dilutions of washed beads (diluted to give about 0.08 or about 0.16 uL as indicated) in PBS (about 20 uL reaction volume) to which about 200 ng pHOT1 supercoiled DNA was added directly, followed by about 30 min incubation at about 37° C. DNA was recovered by proteinase K digestion and analyzed on about a 1% agarose gel. Lane 5 is about 20 uL of W4 (4ₜₕ wash off the column) mixed directly with about 200 ng of pHOT1 supercoiled DNA and incubated as above (lane 6 is a relaxed DNA marker). (B) Cleavage Activity on Beads. Purified topo I cleavages were assayed in free solution form or on beads using about 100 ng pHOT1 supercoiled DNA. The indicated units of topo I were from the washed beads in Panel A (the number of input units of topo I input for the two assay sets were normalized based on relaxation activity.) Proteinase K was used to release DNA for gel analysis. Both solution and bead assays contained about 10 uM CPT added. (C) Cleavage Activity of Bead Bound and Free topo I. Equivalent amounts of topo I activity were titrated on beads (left) or as free enzyme (right) in cleavage reactions containing about 100 uM CPT. The position of nicked open circular (II) DNA is indicated. The plot below shows the digitized data from the gel.

FIG. 3 is a series of images depicting analysis of solid phase and liquid phase Topo I activity. (A) Topo I activity recovery in solid and liquid phase. Topo I titrations were carried out in liquid phase by conventional relaxation assays using about 100 ng of pHOT1 DNA. The solid phase assays were performed by binding the indicated number of ‘liquid based’ units of topo I to plates followed by sufficient washing to remove unbound enzyme (one unit relaxes about 50% of input DNA in about 30 min at about 37° C.). Reactions were carried out in the wells by adding about 100 ng pHOT1 in assay buffer (see “Materials and Methods”) and after incubation, proteinase K (plus about 0.1% SDS final volume) was added and incubation continued for about 15 min at about 37° C. The recovered DNA was then loaded onto an agarose gel (run without ethidium bromide). Supercoiled (form I) and relaxed DNA (form I_{R}) positions are indicated on the right. (B) ELISA Determinations of Bound Topo I. Topo I (specific activity of about 5×10⁶ units/mg protein) was bound and plates washed to remove unbound enzyme. The remaining signal was detected using a monoclonal antibody to topo I.

FIG. 4 is an image illustrating that Topo I cleavages without CPT. Topo I reactions (liquid) were carried out at the indicated input of enzyme with 200 ng of pHOT1 DNA. After termination with about 1% SDS, the reactions were digested with proteinase K and loaded onto an ethidium bromide containing agarose gel. Positions of nicked open circular (II), linear (III) and relaxed (I_{R}) plus supercoiled DNA (I) are indicated (the last two co-migrate in this gel system containing ethidium bromide). The amount of DNA in form II was quantified in each lane in the graph below the gel. The form II DNA was not detected in reactions that were not digested with proteinase K (data not shown).

FIG. 5 is a series of images depicting CPT Induced cleavages in solid phase assays. (A) Solid Phase Activity Titrations. Indicated amounts of topo I (based on liquid reactions) were bound to Nickel plates in triplicate and reactions carried out in the absence or presence of about 5 uM CPT for about 30 min at about 37° C. as indicated. In one set of reactions (about 512 units), the DNA was recovered and analyzed by gel electrophoresis to determine the fraction of relaxed or nicked open circular DNA. As shown, in non-CPT reactions about 80% of the bound DNA was circular relaxed (I_{R}) DNA and about 20% was nicked (II). In CPT containing reactions, relaxed DNA was about 30% and nicked form II increased to about 70%. (B) Fold increase in DNA binding due to CPT. The data in the open boxes are based on results shown in Panel A to reflect changes in CPT stimulation over a range of input WT (wild type) topo I. The black shaded boxes are data from an equivalent titration (in terms of ng input protein) with Y723F topo I mutant that is catalytically inactive but can still bind DNA. (C) DNA binding depends on topo I and DNA. A series of reactions were carried out with the indicated solutes. CPT was added at about 5 uM, DNA at about 100 ng and topo I at about 512 units. Note that backgrounds (+CPT no DNA) were not subtracted (dashed line). (D) Bound DNA is released by DNase I digestion. The reactions were assembled with the indicated components (CPT added at about 5 uM). DNase I digestions were performed at about 50 ug/mL in the wells for about 30 min at about 37° C. and subject to washing steps.

FIG. 6 is a series of images depicting reaction terminations, DNA Inputs and CPT titration data. (A) Effect of SDS in Termination Buffer. Minus (open bars) and plus (about 5 uM) CPT (closed bars) reactions were terminated and washed with the indicated buffers that differ in SDS content. Reactions contained about 512 units of topo I bound to plates. (B) DNA titrations. Increasing amounts of input DNA with about 512 units of bound topo I (minus drug, open bars; plus about 5 uM CPT, closed bars). (C) CPT Titrations. Reactions contained about 512 input units of topo I, about 100 ng of DNA and the indicated CPT concentrations. Identical reactions were processed in solution and the products analyzed by agarose gel electrophoresis with about 0.5 ug ethidium bromide/mL (inset gel).

FIG. 7 is a series of images depicting native and denatured Topo I and test screens in solid phase assay. (A) Native and Denatured Topo I Analysis. Purified topo I (about 512 units) was incubated on ice or at about 95° C. for about 30 min and diluted with PBS (about 1:2, 1:4, 1:8). Each dilution was bound to nickel coated wells as described in “Materials and Methods” using the standard binding reaction (about 100 ng input DNA). After incubation, free DNA was washed out and Picogreen fluorescence was measured. Background signals (no topo input) are shown but were not subtracted (“No Topo”). The inset agarose gel shows the activity associated with the denatured (D) and the native (N) enzyme using the about 1:4 dilution (about 1 uL or about 128 units of topo I). (B) Test Screens. Compounds were selected from the Diversity Set available from the NCI (see “Materials and Methods”) and about 100 uM tested. Reactions contained about 512 topo I units (liquid phase units) and about 5 uM CPT as positive control. Reactions were carried out in triplicate in solid phase. The inset gel shows a parallel set of reactions carried out under identical conditions, except the DNA products were analyzed on an about 1% agarose-ethidium bromide gel to cleanly resolve nicked open circular DNA (II).

FIG. 8 is a flowchart depicting the model for solid phase Topo I screening. His-tag purified topo I is bound to nickel coated plates in a standard binding buffer (typically about 200-500 units). Binding is complete within about 1 hr at room temperature and the reaction is optimized for 96 well plates in a about 100 uL volume. Free enzyme is washed out and pHOT1 supercoiled DNA is added in a topo I assay buffer. After incubating at about 37° C. for about 30 min, all DNA is about 100% relaxed and a fraction is bound to topo I in wells. Free DNA is then washed out and the relative fluorescence is measured using Picogreen staining. In the absence of CPT, the retained DNA is relaxed with some nicked open circular DNA (form II). In the presence of CPT, the retained DNA is mostly form II with a smaller amount of relaxed, protein free DNA. Relative fluorescence (RFU) is shown for a typical reaction with −/+controls (about 2000 and about 4000 fluorescent units respectively). The results are expressed as an “HTS Ratio” corresponding to the RFU of experimental to RFU of negative controls (no CPT).

FIG. 9 is a table depicting the washing and reaction conditions of the method.

FIG. 10 is a table depicting a summary of HTS reconstruction.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that there are other embodiments by which the invention may be practiced. It is to be understood that other embodiments may be utilized and structural changes may be made without departing from the scope of the invention.

According to another embodiment, the invention pertains to a method of detecting agents having topoisomerase modulating activity that involves attaching a topoisomerase to a solid surface such that said topoisomerase retains activity. The topoisomerase is contacted with a polynucleotide (typically DNA) having a sequence recognized by the topoisomerase, in the presence or absence of test agent suspected of modulating activity of the topoisomerase. The modulating effect of the test agent is determined by an increase or decrease of an association of the polynucleotide (fragments thereof) with the topoisomerase.

In a more specific embodiment, the topoisomerase is attached to the solid surface by a tag component, such as a His tag, chitin binding protein (CBP), maltose binding protein (MBP), or glutathione-S-transferase (GST), conjugated to the topoisomerase. In a typical embodiment, the tag is a His tag, and the solid surface is coated with a metal such as nickel or cobalt.

In another related embodiment, the polynucleotide, concurrent to or subsequent to contact with the topoisomerase (in the presence of absence of a test agent), is subjected to a marker compound, such as a dye. An increase or decrease of marker interacting with said polynucleotide, as compared to a control, is indicative of a change in modulation of the topoisomerase. In a typical embodiment, the control is the amount of marker associated with the polynucleotide in the absence of a test agent. It should be noted that the marker would typically be added following a removal of any polynucleotide not associated with the topoisomerase.

In an even more specific embodiment, the marker compound is a fluorescent dye. When a fluorescent dye is used, the method involves comparing fluorescence of the fluorescent dye to a control signal, where the control signal is obtained by contacting only the topoisomerase, the DNA and the fluorescent dye. Moreover, in certain embodiment, an increase in fluorescence indicates a potential interfacial poison and wherein a decrease in fluorescence indicates a potential catalytic inhibitor

According to one example, a method embodiment pertains to a simple method for HTS to identify topo I targeting agents. The technique involves the, binding His-topo I protein to microtiter plates under conditions that preserve enzyme activity. DNA substrate is added and a sensitive DNA dye is used to monitor retention. Reconstruction experiments reveal that topo poisons yield an elevated fluorescent readout, while catalytic inhibitors yield a repressed fluorescent signature relative to no-drug negative controls. The method represents a dual-readout that not only identifies novel topo targeting agents, but also provides relevant insight on mechanism of action. Unexpectedly, it has been discovered that DNA alkylating agents, an important class of genotoxic compounds, can also be picked up by the assay.

In yet another embodiment, the invention is directed to a system for detecting agents having topoisomerase modulating activity. The system involves a solid support having topoisomerase attached to a surface thereof. The topoisomerase is attached to the solid support such that it retains at least some of its activity. In one specific embodiment, the solid support is a microtiter plate. In an even more specific embodiment, the microtiter plate includes a plurality of wells coated with metal. The metal may include nickel or cobalt. The topoisomerase may be bound to the surface via a tag component such as a His tag, and the like. Alternatively, the solid support pertains to a bead.

According to yet another embodiment, the invention pertains to a kit for detecting topoisomerase modulating agents. The kit includes a solid support and a container of tagged topoisomerase. For example, the kit may include a nickel coated microtiter plate, a container of a His-tagged topoisomerase agent, and a container of DNA, wherein the DNA includes a topoisomerase recognition sequence. The kit may further include a container of a dye, such as fluorescent dye.

Supporting Data and Examples

Materials and Methods

Reagents

Anti-topoisomerase I monoclonal and polyclonal antibodies were provided by TopoGEN, Inc. (Port Orange, Fla.). Supercoiled plasmid DNA containing the high affinity topo I hexadecameric recognition sequence (pHOT1) was from TopoGEN. The test compounds were provided by the Developmental Therapeutics Program from the National Cancer Institute as a plated diversity set, a mechanistic set and an approved oncology drug set. The compounds were provided at known concentrations in microtiter plate format. The nickel coated 96 and 384 well plates were from commercial sources (Fisher Thermoscientific). Nickel-NTA agarose affinity beads were Qiagen. Picogreen was obtained from InVitrogen.

Purification of Human Topoisomerase I

Topo I was overexpressed as a His-tagged protein in baculovirus. Experiments were performed with wild type (wt) and mutant (Y723F) proteins from commercial sources (kindly provided by TopoGEN, Inc., Port Orange, Fla.). Results were repeated with enzymes purified as described by Stewart and Champoux. (Stewart L, Champoux J J: Purification of Baculovirus-Expressed Human DNA Topoisomerase I. 1999: 223-234) Spodoptera fuigiperda Sf9 cells were seeded at about 4×10⁷ on a 150 mm dish in Sf-900 Serum Free Media (InVitrogen) supplemented with about 10% FBS (InVitrogen) and infected with high titer virus. Cells were harvested about 72 hr post infection with ice cold about 1× Phosphate Buffer Saline (PBS) and recovered by a low speed centrifugation step (about 400 g for about 5 min). The PBS wash was repeated and the final pellet suspended in 6 mL of homogenization buffer (about 30 mM Tris-HCl pH 7.5, about 4 mM CaCl₂ 1 mM Phenylmethysulfonyl fluoride, about 2 mM DTT, about 5% Sucrose) and incubated on ice for about 15 mins. The cells were homogenized using a tight fitting dounce homogenizer and then centrifuged (about 1200 g for about 15 min at about 4 C). The pellet was suspended in about 7 mL of LB (lysis buffer, about 20 mM NaH₂PO₄ pH 7.4, about 1M NaCl, about 10 mM imidazole and EDTA free protease inhibitors from Roche) and incubated for about 30 min (ice) followed by addition of about 3 mL LB containing about 18% polyethylene glycol (PEG). The solution was incubated on for about 30 min on ice and centrifuged for about 30 min (about 40,000 g) at about 4° C. The supernatant recovered and mixed with Ni—NTA Agarose beads pre-equilibrated with the about 20 mM NaH₂PO₄ pH 7.4, about 1M NaCl and about 10 mM imidazole followed by overnight gentle rocking incubation step at about 4° C. The slurry was then placed in a small polyprep chromatography column (Bio-Rad) and allowed to settle (about 15 min). The column was then washed with about 30 mL of Wash Buffer (about 20 mM NaH₂PO₄ pH 7.4, about 300 mM NaCl and about 20 mM Imidazole) followed by a series of about 1 mL washing steps with Elution Buffer (about 20 mM NaH₂PO₄ pH 7.4, about 300 mM NaCl and about 250 mM Imidazole). Proteins were detected by absorbance at 280 nm and concentrations determined by Bio-Rad Protein assay using BSA as the standard. Samples were also subsequently analyzed on about 10% SDS-PAGE gel and stained with Gel code Blue Stain reagent (ThermoScientific). Protein containing fractions were pooled and the imidazole removed by dialysis against about 700 mL of about 20 mM NaH₂PO₄ pH 7.4, about 300 mM NaCl, about 10% glycerol, about 0.5 mM DTT. The topo I active fractions were supplemented with about 50 μg/ml BSA prior to dialysis, to help stabilize the activity. Topo I purity was greater than about 98% and was stored at about 4° C. for up to about 6 months without loss of activity. The final purity was checked by SDS-PAGE analysis of overloaded gels and activity assays confirmed high levels (>1000 units per ul) of topo I. The purified fraction was free of topo II as determined by kDNA decatenation analyses and Western blot probings using anti-topo II polyclonal antibody (TopoGEN, Inc.). The final fraction was nuclease free based on incubation of pHOT1 with excess (>500 units) of topo I in the presence of about 5 mM MgCl₂ and testing for the formation of nicked, open circular DNA (form II) or linear DNA (form III). One unit of topo I will relax approximately about 50% of pHOT substrate (about 100 ng input) in about 30 min at about 37° C. The final specific activity of a typical preparation ranged between about 0.5 to about 5.0×10⁶ units per mg of protein (total yield of about 2 mg).

Plasmid Relaxation Assays

Topo I was assayed by relaxation of pHOT1 supercoiled DNA (form I). Reactions were carried out in TGS buffer (about 10 mM Tris HCl (pH8.0), about 1 mM EDTA, about 150 mM NaCl, about 5% glycerol, about 0.1% BSA and about 0.1 mM spermidine) and about 100 ng form I pHOT1 DNA for about 30 min at about 37° C. For titration analyses, the enzyme was diluted two fold and about 1 uL was assayed from each dilution step in a final reaction volume of about 25 uL. Reactions were terminated with about 5 uL of stop buffer (about 5% sarkosyl, about 0.125% bromophenol blue, about 25% glycerol) and loaded onto a about 1% agarose gel. The gels were run at about 1.5-2 V/cm until the dye front was about 75% down the gel, followed by staining for about 30 min with about 0.5 ug/mL ethidium bromide (EB), destaining for about 10 min in water and digital imaging using a Gel Doc system (Syngene).

Cleavage Complex Formation

Topo I cleavage assays were performed in the presence and absence of known positive topo I active drugs or with test drugs. Reactions were incubated at about 37° C. for about 30 min and terminated by addition of SDS (about 1% vol/vol final) followed by digestion with about 0.5 ug/mL proteinase K (about 30 min at about 56° C.). The DNA was extracted by Phenol: Chloroform precipitation, using standard methods and following addition of about 3M sodium acetate, about pH 5.2 (about 0.1 vol), about 2 uL of about 20 mg/mL glycogen, the DNA was ethanol precipitated. The pellet was washed with about 100 uL of about 70% cold ethanol, air dried and then dissolved in about 20 uL of TE (about 10 mM Tris-HCl, about pH 7.5, about 1 mM EDTA). The DNA was then subjected to electrophoresis on about 1% agarose gel containing about 0.5 ug/mL of ethidium bromide (in gel and running buffer).

This gel system clearly resolves form II DNA (nicked open circular DNA) from circular forms (supercoiled, form I and relaxed; form Iᵣ). In some cases, DNA samples were divided in equal parts for analysis in non-EB gels (resolves form I and Iᵣ) and EB gels (resolves form II topo I cleavage products). All gels contained appropriate markers for unambiguous assignment of topological or cleavage status.

High Throughput Assays

The HTS assays were performed in the microtiter well format (96 or 384); however, most of the data shown are based on the 96 well format. A fixed number of topo I units in a final volume of about 50 uL were bound to nickel coated plates for about 2 h at room temperature (multiple incubations and temperatures were tested and these conditions gave optimal binding). The unbound enzyme was removed by aspirating off the initial binding solution followed by three washes (about 200 uL each) with cold PBS containing about 0.05% Tween-20 (PBS-T). (These conditions were demonstrated to remove all unbound topo I as determined by activity assays in the washes and Western blotting with topo I monoclonal antibody probe.) Topo I reactions were initiated by addition of a pre-mix solution of TGS, about 100 ng of pHOT1 DNA in the presence or absence of test or control drugs. Drugs were dissolved in DMSO and the final DMSO concentration in the reaction never exceeded about 1%. Reactions were incubated for about 1 hr at about 37° C. and terminated by the addition of about 0.1 vol of about 1% SDS (vol:vol) followed by a about 5 min incubation at about 37° C. The reaction mixture was next aspirated and washed three times with about 200 uL PBS-T. Picogreen (about 100 ug/mL) was diluted in TE (about 1:400) and about 200 □l was added to each well followed by incubation in the dark for about 5 min. The relative fluorescence was measured at about 485 nm excitation and about 525 nm emission wavelength using a Tecan reader.

ELISA

To measure amounts of bound topo I (antigen), ELISA was used with an anti-topo I antibody (provided by TopoGEN, Inc.). The primary antibody was diluted about 1:1000 in PBS-T and about 100 uL added per well. After 1 hr incubation with primary antibody, the wells were washed three times with about 200 uL of PBS-T, followed by the enzyme conjugated secondary (rabbit anti-mouse, TopoGen) at about 1:500 in PBS-T. Plates were washed three times again with PBS-T and quantified at about 595 nm using TMB Peroxidase EIA substrate kit (Bio-Rad).

Data Analysis

Z′ factor analyses were performed to determine: dynamic range and variability of the topo I HTS. (Zhang J-H, Chung T D Y, Oldenburg K R: A Simple Statistical Parameter for Use in Evaluation and Validation of High Throughput Screening Assays. Journal of Biomolecular Screening 1999 4:67-73) Z′ values should exceed about 0.5 for a functional and robust HTS assay based on equation (1). HTS experiments were conducted and the data analyzed from about 10 different experiments performed on different days with different operators and using different inputs of topo I as well as different lots of enzyme and CPT.

$${Z}^{′} = 1 \cdot \frac{3 ( SD of + CPT Well + SD of \cdot CPT Well )}{Mean of + CPT Well \cdot Mean of \cdot CPT Well} \qquad ( 1 )$$

### Results

Topo I Overexpression in Baculovirus as a High Yield HTS Source.

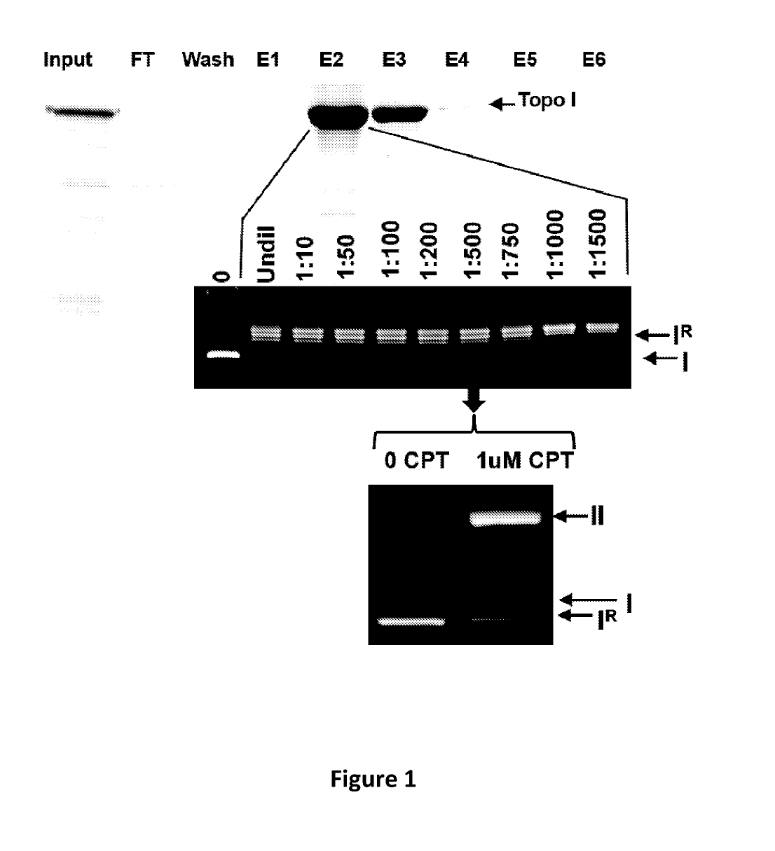

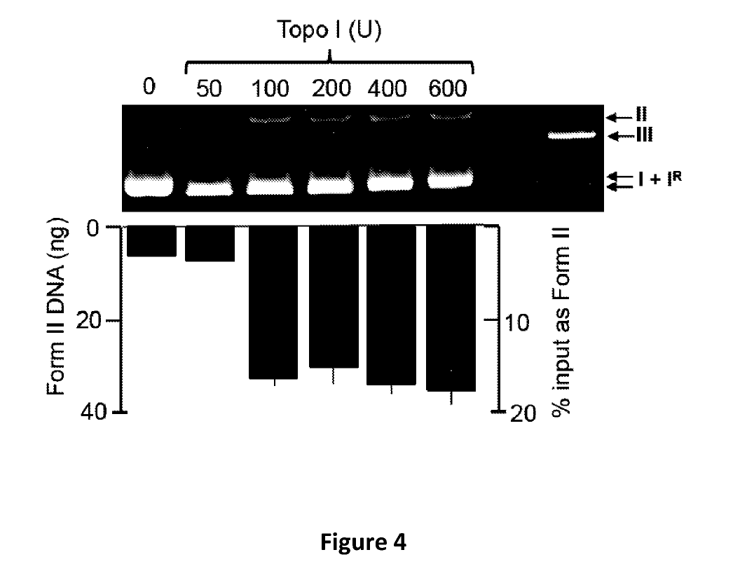

The intact human topo I gene was cloned as a His-tag gene for expression in Baculovirus (see “Materials and Methods”). Other tags such as GST tags may also be used. Immobilized metal affinity column chromatography using a nickel column resin was to purify the enzyme to homogeneity (FIG. 1). High yields of intact topo I (mg of protein) with total activity levels typically well over two million units where consistently obtained. The assay data show a routine preparation of more than about 1500-2000 units per uL (see upper gel inset FIG. 1); therefore, topo I was highly active and suitable for HTS operations of scale. The purified enzyme was capable of detecting topo I cleavage products both in the presence of CPT (lower gel inset, FIG. 1) and (with higher inputs) in the absence of drug (FIG. 4B). The intact, about 100 kDa form of topo I, was stable. Under the experimental conditions used, degradation to catalytically active lower molecular weight forms was not detected (data not shown).

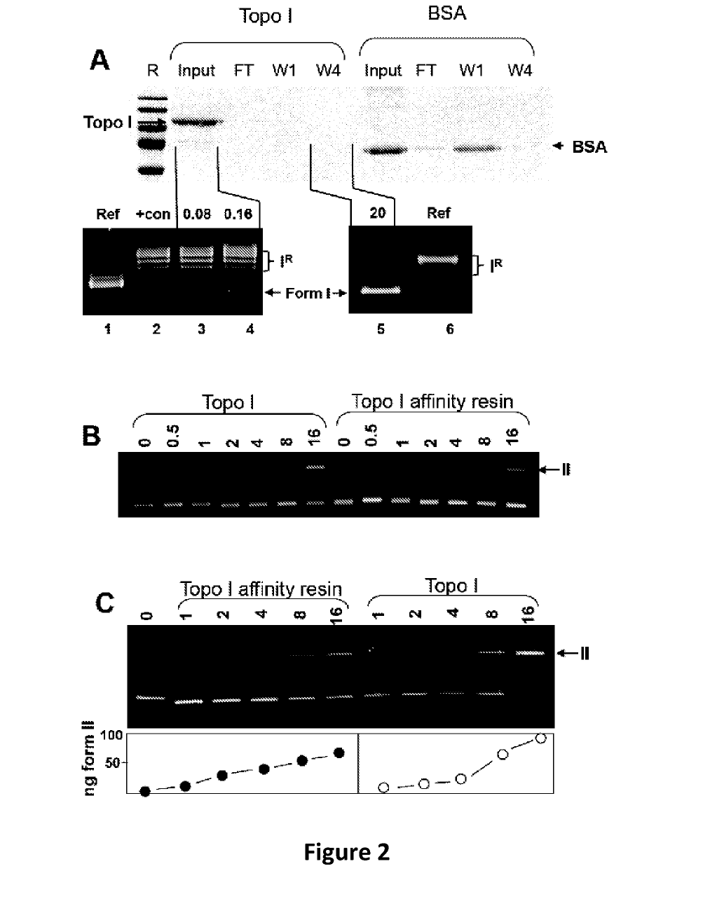

Since the goal was to bind active topo I to a solid phase, it was next next determined whether nickel bound topo I retains activity. Topo I was bound to a nickel affinity column and a heterologous protein (bovine serum albumin) was compared as a negative control. As shown in FIG. 2A, all input topo I bound the column and none was detected in the column void and flowthrough fractions. To ensure that no residual unbound topo I might be present the multiple washes (wash #1 and #4 shown, panel A) were performed and relaxation activity in the last column wash was directly assayed (W4). Compared to input topo I (that went onto the column) topo I activity with over about 250 fold greater volume as compared to input was not observed. Coomassie blue staining of wash fractions did not detect any topo I polypeptide (note that more sensitive Western blots were similarly negative, data not shown). In contrast, BSA was readily detected in the column void and wash #1, however essentially all of the BSA was desorbed out after about 4 washes; thus, our column washing methods effectively remove traces of unbound protein. The washed resin with bound topo I was then incubated with supercoiled plasmid DNA and challenged with CPT to determine if cleavage complexes could be detected. Parallel reactions with the same topo I preparation, except in solution, were carried out as a control.

The data show that the resin bound topo I retained excellent CPT mediated DNA cleavage activity (FIG. 2B, 10 uM CPT and 2C, 100 uM CPT). The activity titration curves appear different (FIG. 2C) in that the resin bound topo I displayed a uniform increase in form II DNA product (nicked open circular) while the titration in solution appeared to be sigmoidal; however the differences were not large. In addition, the liquid reactions were more robust overall with a total yield of DNA cleavage product approaching about 95 ng DNA compared to about 65 ng for the resin bound enzyme (digitized data below FIG. 2C). It is estimated that trapping the enzyme on a resin reduced the cleavage efficiency by about 20-30% for a given unit input of topo I. Note that in these reactions, about 100% conversion of supercoiled DNA to cleavage product was not seen; therefore, the reactions were carried out in the linear range. These data establish that topo I bound to a nickel agarose bead retains cleavage and relaxation activity with a about 20-30% reduction in overall efficiency.

Immobilization of Topo I in Solid Phase and Activity Recovery

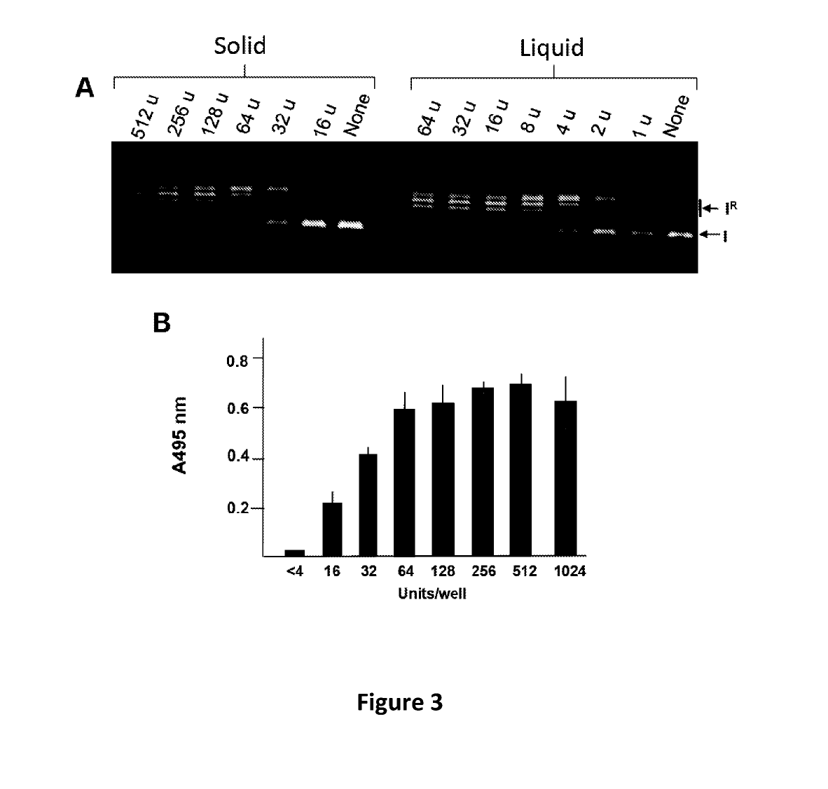

To investigate topo I activity on a solid surface, the enzyme was bound to 96 well nickel coated plates and the wells were extensively washed. Reconstruction experiments on washing conditions establish that even with very high (about >2000 units) input of topo I, three washes were sufficient to remove unbound topo I and reduce activity to undetectable levels in the last wash (data not shown but see FIG. 2A). To facilitate comparisons, the same lot of enzyme was used and equivalent amounts of activity were put in the wells. Supercoiled DNA was added to each well and topo I relaxation activity of the bound enzyme compared to the same unit concentration of topo I in a standard liquid assay was measured. By comparing product yields for both reactions, it was concluded that the affinity binding of topo I reduced the activity by approximately 90% or more (compare ‘solid’ and ‘liquid’ relaxation data, FIG. 3A).

Two-fold dilution gel based assays are not very quantitative however, in the liquid phase assays, full relaxation required about 4-8 units of enzyme. In solid phase, this level of activity required about 64-128 units of activity. Taking average values, about 6 units in liquid were required to fully relax the plasmid versus about 92 units in solid phase. This indicates about a 6.2% recovery of active enzyme in the tethered state (or about a 94% loss of activity between liquid and solid assay states). In repeat experiments, the loss was in the about 80-95% range. Some of this loss may be due to inactivation during the binding incubation period and presumably to trapping of enzyme in a form that does not support enzymatic activity; however, it is suspected that most of the loss may be caused by pinning the enzyme to a solid surface which either affects controlled rotational events or limits diffusion rates and the ability to engage the DNA in a three dimensional search. The latter prospect seems likely since large activity losses were not observed when binding topo I to an affinity bead (FIG. 2) which is free to move through the solution reaction.

While about >90% loss of enzyme activity appears to be extreme, if the solid phase assay and topo I yields are sufficiently robust, such a loss need not be a rate-limiting step. To address this point, it was next determined determined how much enzyme each well could accommodate at saturation. Nickel coated microtiter wells were tested to assess binding efficiency. The wells were hydrated using a solution of PBS-T (PBS with and about 0.05% Tween) producing stringent conditions for specific binding (however, these conditions are compatible with topo I activity, not shown). Different concentrations of enzyme were diluted into about 50 ul of PBS-T and added to each well followed by incubation for about 2 h at room temperature with mild shaking. Topo I binding was confirmed by ELISA using a mouse monoclonal primary antibody specific for topo I (FIG. 3B). Maximum protein binding was detected with about 256-1024 units corresponding to about 0.25 to about 0.5 ug of input topo I. These values are very close to the theoretical maximum of binding capacity of the wells, based on information from the commercial supplier of the plates. To more accurately assess saturation binding, the topo I was eluted (with imidazole) and the recovered protein was analyzed by Western slot blotting (which gives good linearity with higher concentrations of antigen).

Based on slot blots, the binding capacity is actually closer to about 1.5 ug of enzyme (data not shown). This result suggests that each well can accommodate large amounts of topo I and despite losing about 85-95% of the input activity, sufficient amounts of enzyme can be bound to over-ride this limitation.

Topo I Cleavages in the Absence of IFP.

As described below, this HTS requires relatively high input topo I levels; therefore, determination as to if cleavages occur and to what extent the input DNA is converted to cleaved product is needed. The data in FIG. 4 show that some nicked cleavage products accumulate even in the absence of CPT. At a threshold level of about 100 units of input topo I, cleavage product formation can be seen in liquid assay. The amount of form II cleavage product did not significantly increase even at much higher ratios of enzyme: DNA (up to about 600 units of topo I) and the cleavages reached a stoichiometric maximum at about 100 units. That the cleavage product was due to topo I and not a contaminating nuclease can be ruled out since the form II DNA was not detected unless proteinase K digestions were carried out (topo I/DNA complexes do not enter the agarose gel and are ‘gel shifted’, data not shown). This result clearly demonstrates that topo I cleavage complexes form in the absence of CPT, especially at high input levels of enzyme. Moreover, the cleavage reactions are not saturated since only a small fraction of input pHOT1 DNA (less than about 20%) was converted to form II product (FIG. 4, histogram). From this observation, detecting novel IFPs should be possible since there is a large pool of uncut DNA substrate. Note that linear DNA (form III DNA) with high topo I input was not detected; therefore, the possibility that nested topo I cuts on opposing strands would spontaneously form a cleavage complex can be ruled out. (FIG. 4, inset gel).

Detection of CPT Mediated Cleavage Complexes in the Solid Phase Assay

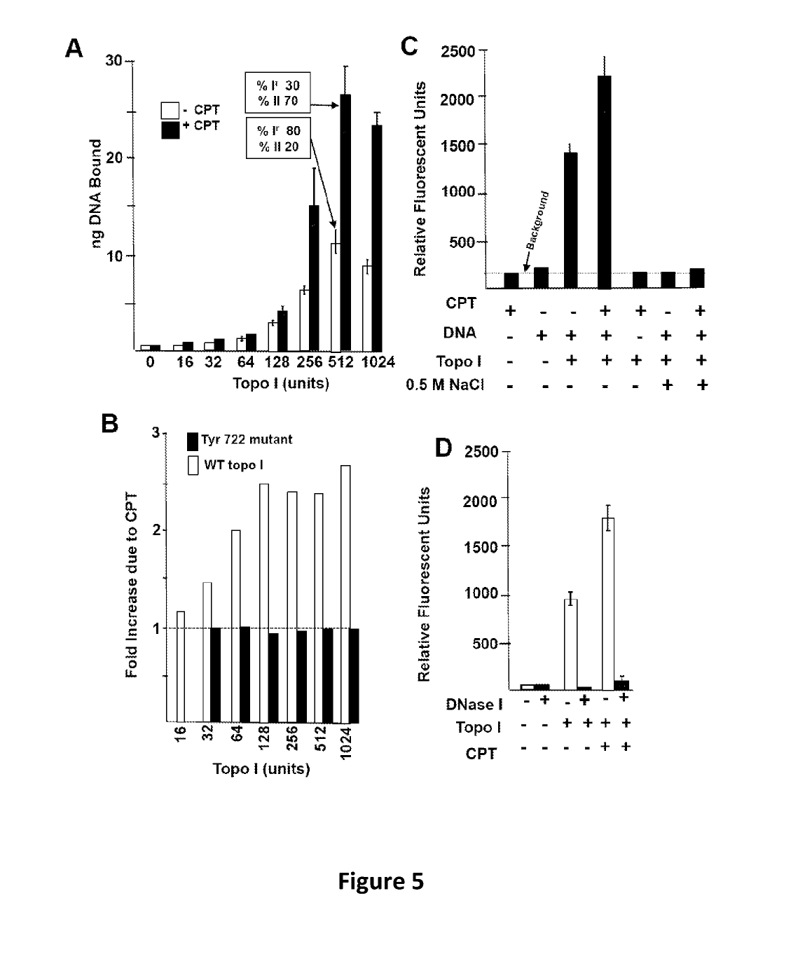

Next, topo I was titrated in the presence and absence of a prototypic IFP (CPT) using topo I bound to wells in the solid phase format. At low concentrations of topo I, signals were low as expected; however, at all concentrations tested, the fluorescent signature was greater in the presence of CPT (FIG. 5A). Note that CPT stimulation increased with more input topo I; thus, between about 16-64 units, CPT resulted in about 1.2-2 fold increases in DNA binding to wells, while reactions containing about >100 units gave about 2.5-2.7 fold increases. This behavior is consistent with the conventional liquid assays (i.e., cleavage complexes require higher inputs of topo I since cleavages are not catalytic, for example see FIG. 4). To demonstrate that CPT stimulation was correlated with topo I activity, a catalytically inactive mutant (Y723F) was prepared that can bind DNA (but cannot initiate cleavages). The mutant protein did not display any CPT stimulation at the highest input of protein (shaded black boxes, FIG. 5B); however, DNA binding (relative fluorescence) was detected with mutant topo I, as expected (data not shown).

The DNA retained in wells (after reaction termination) was also analyzed by digesting with proteinase K to release the bound DNA reactants, and running an agarose gel to resolve relaxed (Iᵣ) and nicked open circular DNA (form II). In the absence of CPT (FIG. 5A, about 512 units) about 20% of the DNA that was bound to wells was form II (and about 80% Iᵣ). In CPT reactions, form II DNA increased to about 70% (about 30% Iᵣ).

Based on these results, the solid phase assay can detect interfacial poisons. Additional controls verify that the signals that were detected depend on presence of all reactants in the wells. CPT alone or DNA alone (FIG. 5C) gave background signals (note that CPT can fluoresce and these data show that it does not contribute to the readout). Similarly, adding CPT to topo I without input DNA gave background fluorescence. The Picogreen signal is seen only in the presence of topo I and DNA and that signal is enhanced in the presence of CPT. Finally, the Picogreen signal is lost when the wells are digested with DNase I; however, a small background remains in CPT reactions because topo I partially blocks access of the nuclease in the covalent complex, protecting about 25 by of DNA (FIG. 5D). (Trask D K, Muller M T: Biochemical characterization of topoisomerase I purified from avian erythrocytes. Nucleic Acids Research 1983 11:2779-2800)

In order to determine the potential ability of the solid phase assay to detect agents that inhibit topo I/DNA interactions (CICs), the effects of increasing ionic strength in the reactions was tested. Salt concentrations above about 0.25M do not favor topo I/DNA binding, a necessary antecedent step toward covalent complex formation. As shown in FIG. 5C, high NaCl essentially eliminates DNA binding; therefore, the solid phase HTS assay is responsive to agents or conditions that reduce DNA binding. This result demonstrates the potential for the solid phase HTS to detect agents that disrupt the ability of topo I to engage DNA.

Termination and Optimization of Solid Phase Reactions

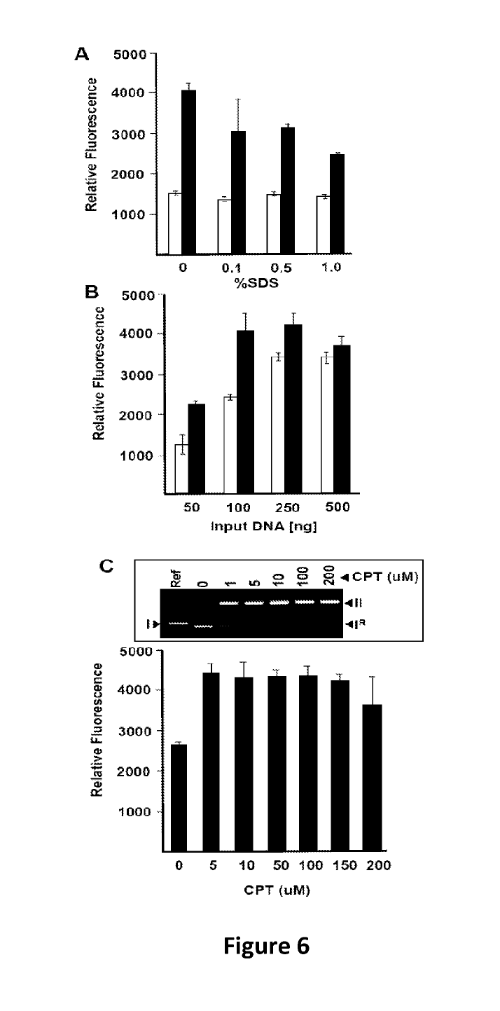

Termination and washing conditions of the solid phase reactions were examined next. The presence of SDS was examined to evaluate how denaturation of topo I, which is normally an excellent method for trapping cleavage complexes, might influence solid phase assay results. SDS reduced the CPT complexes (about 10-30% without affecting the non-CPT residuals) and the impact of this SDS decrease was nearly identical from about 0.1 to about 1% (FIG. 6A). SDS in this range does not disrupt the nickel-His affinity complexes₁₃ and it was confirmed that topo I is not being released from the plates under these conditions (data not shown). From this result the it was concluded that SDS reduces CPT based signals but has little if any affect on non-CPT reactions.

Increasing DNA inputs resulted in an increase in DNA complexes for both drug and no drug reactions (FIG. 6B) and it is economical to use lower amounts of DNA; therefore, about 50-100 ng of input supercoiled DNA is ideal. Moreover, higher levels of input DNA reduce the difference between minus and plus drug results, which is not ideal for detecting IFP agents. The influence of elevated CPT concentrations was evaluated and it was found that the solid phase assay did not display drug concentration dependency (FIG. 6C). When drug dependent cleavage results were compared with conventional solution reactions (at high topo I inputs as used in solid phase assays), strong drug concentration dependency (inset gel, FIG. 6C) was not observed probably due to relatively high topo I: DNA ratios (ca. 100:1); thus, each form II molecule may incur more than one single nick. Since appearance of form II DNA is a single hit phenomenon, high enzyme ratios favor efficient formation of the nicked cleavage product, even at relatively low levels of CPT; however, this result is an advantage in HTS and enhances sensitivity of the assay (favors the detection of weak IFP activities).

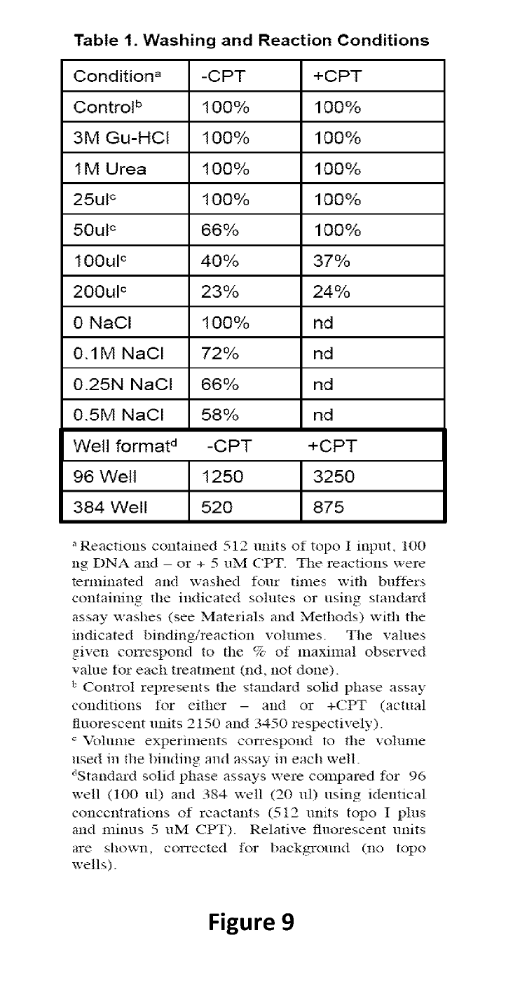

The different termination and washing procedures were evaluated to develop some understanding of the nature of DNA retention once the input plasmid engages the attached topo I (FIG. 9). Chaotropic agents (urea, guanidium hydrochloride) had no affect on DNA retention either in presence or absence of CPT. Increasing the volume of the binding/reaction in a 96 well plate format reduces the fluorescent signal somewhat, probably because surface areas increase to a larger extent over volume, thereby decreasing the probably of productive topo I/DNA interactions, as noted above. Terminating and washing steps with elevated salt reduce tends to release some of the bound DNA (but not all) and even very high NaCl levels (about 1M-2M) do not release the DNA once it is bound (note that high salt will prevent binding, FIG. 5C).

The the standard solid phase assay in a small volume 384 well format was tested. The magnitude of the increase in +CPT controls was less obvious (about 1.7 fold compared to about 2.6 fold), and it was concluded that the assay is adaptable and functional in 384 well plates.

Heat Denatured Topo I is Inactive in Solid Phase Assays.

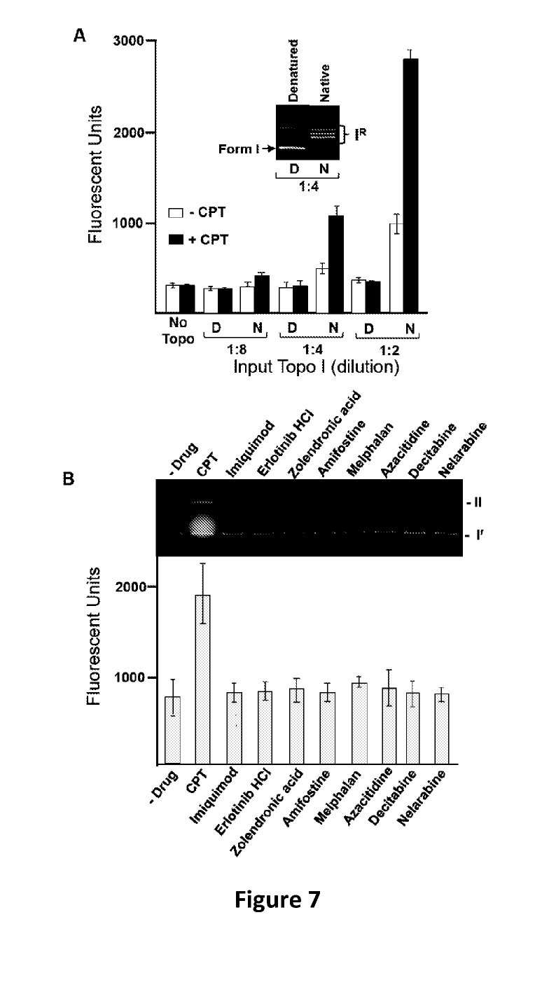

To examine topo I activity and DNA binding, the enzyme was heat denatured before binding to affinity wells and compared the results with the identical preparation of native enzyme. The denatured protein bound efficiently to the wells using ELISA (as in FIG. 3, not shown) and the denaturation step effectively inactivated the enzyme (see inset gel FIG. 7A). DNA retention in CPT and non-CPT reactions containing identical dilutions of the enzyme was then measured (FIG. 7A). The data clearly show that CPT stimulation requires catalytically active topo I in particular at higher inputs of topo I (noted above). In the no drug reactions, significant increases in DNA retention of native over denatured enzyme were observed, especially at the higher inputs of topo I (see 1:2 and 1:4 dilutions, FIG. 7A).

Reconstruction of topo I HTS.

From the ‘approved oncology drugs set’ available from NCI (Developmental Therapeutics Program), 8 compounds were selected at random for reconstruction testing of the solid phase HTS Assay. These agents were tested at a relatively high concentration (about 100 uM) in order to assess whether the assay is influenced by non-specific events. The tested drugs range from DNA hypomehtylating agents (Azacitidine, Decitabine), tyrosine kinase inhibitor (Erlotinib), a topo II catalytic inhibitor and radio-chemoprotective agent (Amifostine), an immune response modifier (Imiquimod), a bifunctional alkylator (Melphalan) and a bisphonic acid that inhibits bone resorption (Zoledronic acid). All of these agents inhibit cell growth with IC50 in the low micromolar range and except for Amifostine, are non-topo targeting agents.

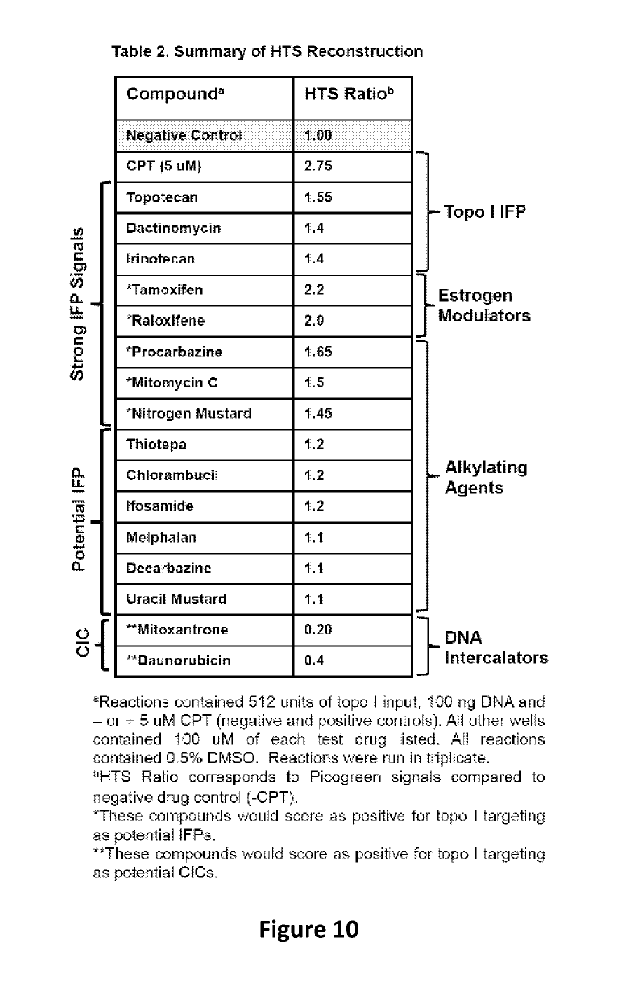

The positive and negative controls (left two most bars in histogram, FIG. 7B) set the high and low parameters for the bimodal readout for IFCs or CICs respectively. None of the agents would be scored as either IFPs or CICs based on solid phase HTS. Moreover, the tested compounds did not influence topo I activity (see inset gel, FIG. 7B). The alkylating agent Melphalan yielded an elevated readout (Melphalan/no drug control ratio of about 1.2-1.3). While this is not a large increase, it suggested that alkylators related to nitrogen mustard agents may give an intermediate readout that is less than the +CPT control. Other alkylators, as well as other drugs using the HTS, assay were therefore tested (FIG. 10). A total of about 50 purified drugs were screened at a high (about 100 uM) concentration. These data confirm that alkylating agents score an intermediate readout (with about 1.0 being the minus CPT control). Alkylating drug readouts ranged from about 1.1 (Uracil mustard) to about 1.65 (Procarbazine). Estrogen modulators (Tamoxifen and Reloxifene) gave particularly high ratios (about 2.2 and about 2.0 respectively). Tamoxifen has been reported to be a topo I poison which explains the HTS results (FIG. 10). (Larosche I, LettÃ©ron P, Fromenty B, Vadrot N, Abbey-Toby A, Feldmann Gr, Pessayre D, Mansouri A: Tamoxifen Inhibits Topoisomerases, Depletes Mitochondrial DNA, and Triggers Steatosis in Mouse Liver. Journal of Pharmacology and Experimental Therapeutics 2007 321:526-535). In addition, other topo I IFPs gave intermediate levels (HTS ratios about 1.4-1.55) and would have been scored as positives. Intercalating drugs were strongly inhibitory and pushed the ratios to low levels, suggesting that strong DNA intercalators are inhibiting DNA/topo I interactions. (Wassermann K, Markovits J, Jaxel C, Capranico G, Kohn K W, Pommier Y: Effects of morpholinyl doxorubicins, doxorubicin, and actinomycin D on mammalian DNA topoisomerases I and II. Molecular Pharmacology 1990 38:38-45).

Z′ Determinations

A useful parameter to assess signal dynamic range as well as control variations is the Z′ factor. (Zhang J-H, Chung T D Y, Oldenburg K R: A Simple Statistical Parameter for Use in Evaluation and Validation of High Throughput Screening Assays. Journal of Biomolecular Screening 1999 4:67-73). Z′ values for the data for assays were determined that display maximal differences between positives (+CPT) and negative drug controls. For example, about 1024, about 512 unit input reactions (FIG. 5A). The +CPT mean about 2877 (SD +/− about 153) and in −CPT the mean value was about 1181 (SD +/− about 44.5) for high topo I inputs for a Z′ value of about 0.808. These data were derived from multiple experiments performed on different days using different lots of enzyme and with different operators. Thus, the solid phase method is robust in detecting IFP active agents like CPT (Z′ values in excess of about 0.5 are considered acceptable). (Zhang J-H, Chung T D Y, Oldenburg K R: A Simple Statistical Parameter for Use in Evaluation and Validation of High Throughput Screening Assays. Journal of Biomolecular Screening 1999 4:67-73) Z′ values were strongly dependent on topo I inputs, as expected, since the differences between positive and negative controls was much less obvious below a certain threshold of input enzyme. Experiments with low input topo:DNA ratios yielded a lower CPT stimulation index (see FIG. 5A, 16-64 unit); however Z′ values were still greater than about 0.5 (about 0.57 to about 0.58).

Discussion

The Topo I HTS System.

It is well established that topo targeting agents represent potential anti-cancer therapeutics and progress in finding new drugs would be enhanced with tractable HTS technologies that exploit new systems for over-expression, streamlining and automating the process. Most of the prior HTS strategies for topoisomerases have focused on analysis of the DNA products (structural or topological changes affiliated with enzyme action) using physical detection methods. (Shapiro A, Jahic H, Prasad S, Ehmann D, Thresher J, Gao N, Hajec L: A Homogeneous, High-Throughput Fluorescence Anisotropy-Based DNA Supercoiling Assay. Journal of Biomolecular Screening; 15:1088-1098; Maxwell A, Burton N P, O'Hagan N: High-throughput assays for DNA gyrase and other topoisomerases. Nucleic Acids Research; 34:e 104-e 1 04)

Others established this concept early on with microtiter-based assay and eukaryotic topo II. (Muller M T, Helal K, Soisson S, Spitzner J R: A rapid and quantitave microtiter assay for eukaryotic topoisomerase II. Nucleic Acids Research 1989; 17:9499-9499). Such HTS methods have good potential to quickly gate out agents that alter topo I functional interaction with DNA.

In the current work, a relatively simple strategy to detect and quantify topoisomerase action in an HTS application was tested that involves active enzyme on a solid surface. By crafting the assay in this manner, a diffusion limited solution reaction was converted to a solid phase detection method, amenable to high volume automated processing with a mechanistic readout. Tethering the ligand (topo I) to a solid surface imposes restrictions on its ability to efficiently interact with the DNA target; thus, the efficiency is adversely affected and the enzyme activity is reduced almost about 10 fold in the bound vs. free state. This is not as serious as it may seem since the topo I system yields are extremely robust and amenable to HTS operations. Specifically, this method will detect interfacial poisons (IFP's) as well as agents that interfere with the ability, of the enzyme to engage the substrate (catalytic inhibitor compounds or CIC's).

A Model for Solid Phase Topo I HTS

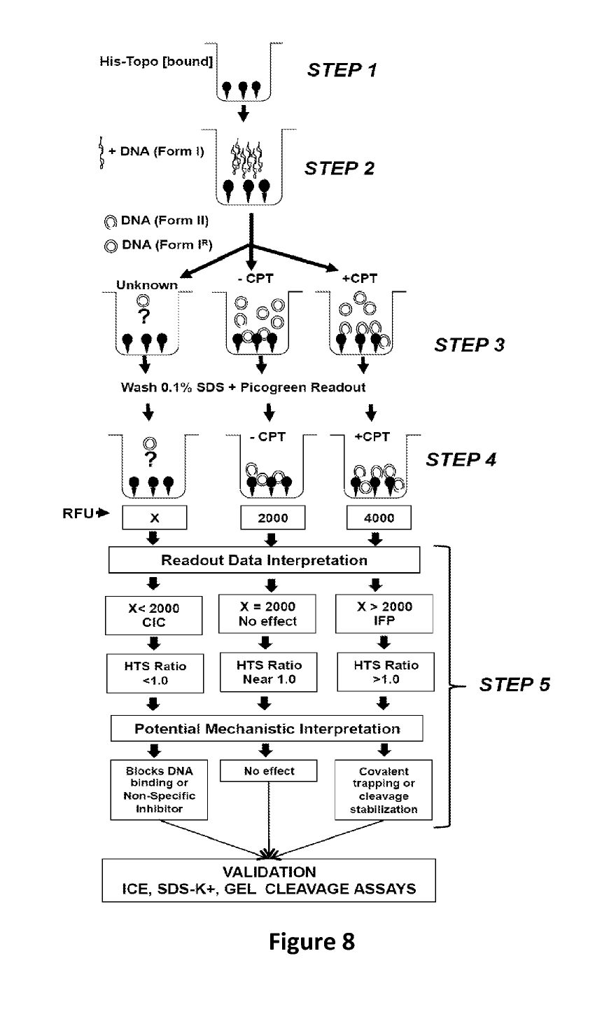

A model that describes our findings with the topo I solid phase assay is presented in FIG. 8. Each step in the model and the pieces of supporting evidence for this model are as follows.

First, His-tag topo I binds to a nickel coated plate and free protein is washed out (steps 1 and 2). The initial experiments demonstrated convincingly that a monomeric protein like topo I retains DNA binding and catalytic function when bound to nickel coated beads. This ‘hybrid’ experiment (beads in solution) revealed that topo I action was reduced by about 20-30% (FIG. 2).

Second, supercoiled DNA is added to the wells under conditions optimal for topo I activity and cleavage (step 3). Topo I bound on the surface of the wells is active in both cleavage and relaxation; however, there is a substantial loss in enzyme activity. This was determined by binding topo I to the wells and performing sufficient washes to effectively eliminate unbound topo I. By adding supercoiled DNA to the wells directly, the ability of bound topo I to relax the substrate can be assayed. The bound enzyme was approximately 80-90% less active in relaxation compared to the free topo I in solution. Desorption or degradation of topo I in the wells can be ruled out, since the protein is intact and not released after incubating with plasmid DNA (the intact polypeptide was recovered from the wells after the reaction, data not shown). In addition, when topo I was incubated in the wells without DNA, then the overlay solution recovered (depleted in topo I) and assayed for relaxation, no topo activity was detected since the enzyme was efficiently bound to wells.

Finally, the reaction and washing conditions are compatible with binding of a typical His-Tag protein. (Loughran S T, Walls D: Purification of Poly-Histidine-Tagged Proteins. Protein Chromatography:311-335) One possible explanation for the lower efficiency in solid phase is that the ability of topo I to interact with DNA is limited when the protein is pinned down to the surface. Two pieces of data are consistent with this view. First, the CPT trapping shows an unusual kinetic signature. The inventors found that CPT induced complexes in solid phase continue to accumulate over several hours, while in solution the complexes reach a maximal stoichiometric value within a few minutes. Thus, it seems that limiting enzyme diffusion through the reaction mixture compromises the ability of topo I to make productive cleavage contact with the DNA. Second, this idea would suggest that smaller volumes might give better picogreen signals with a fixed concentration of topo I. This appears to be the case although the affect is not large (FIG. 9). These considerations suggest that smaller scale binding/assay would work. Indeed, the solid phase assay can be adopted to a smaller well although CPT stimulation was a bit less than the 96 well assay as were overall fluorescent readouts (FIG. 9). Despite the large (ca. 10 fold) loss of activity, the system described is well suited for HTS operations. For example, a single enzyme preparation yields sufficient topo I to screen about 5,000-6,000 compounds (96 well format) at about 500 units/well and about 10,000 to about 12,000 compounds about 250 units/well (which also works, see FIG. 5A).

In Step 3 (FIG. 8) topo I reaction products, both as relaxed DNA and nicked open circular DNA are detected by picogreen. In the presence of CPT, DNA binding is elevated compared to negative drug controls since cleavage complexes are retained. This is a highly reproducible observation that is eliminated if the enzyme is denatured (FIG. 7) or with a catalytically inactive mutant topo I (FIG. 5B). It was noted that the magnitude of the CPT effect is amplified with higher topo I inputs. The most likely explanation here is that cleavage requires stoichiometric amounts of topo I and each complex consumes one topo I molecule; thus, higher ratios of topo I:DNA favor trapping of cleavage complexes (which is true in solution based assays as well). When the inventors analyzed nicked or relaxed DNA status in the wells, the results were consistent with what one would expect (FIG. 5A); viz., form II DNA was clearly increased in +CPT wells over no drug controls. In addition, the magnitude of the increase in form II DNA was commensurate with the increase detected in the solid phase assay (e.g. about 2-3 fold).

Fourth (step 4, FIG. 8), it is noted that all of the bound DNAs are topo I reaction products (linears or supercoiled DNAs were not detected); thus the picogreen signals are detecting topo I reaction products in the wells. Retention of form II DNA in the wells is due to formation of the cleavage complex, a stable, protein-DNA intermediate. In step 5, the readout (relative fluorescent units or RFU retained in the wells) is analyzed based on what is referred to as the ‘HTS Ratio’ (RFU Experimental/RFU in negative control).

There are in fact two reference values that are important in data interpretation: the HTS Ratio and the positive control (+CPT). The former predicts the mechanism of test drug action (IFP vs. CIC) and the latter validates that that all components in the HTS screen are working as expected (enzyme, DNA, buffers, etc.). The positive control also serves to demonstrate solvent effects on the results, an important consideration. If the HTS Ratio is less than unity, it is concluded that a CIC has been identified. HTS Ratios near unity would be ignored and HTS Ratios greater than unity would be scored as either IFP or as DNA damage agents. These ‘on target’ vs. ‘off target’ outcomes are easily distinguished by simple agarose gel assays of the hits. Conservatively, it is estimated, based on reconstruction experiments with purified compounds (FIG. 10, FIG. 7) that the HTS will eliminate about 95-98% of the compounds; therefore, in a about 25,000 compound screen about 500 to about 1000 hits could be detected (this is probably an overestimate). Any HTS ratios that show extreme values (i.e., about 0.2 or about >2) would be of immediate interest for follow-up studies (it is estimated that less than about 10 hits per about 25,000 compounds tested). Follow-up secondary screens in most cases would be quite simple, for example, testing whether the compound nicks a plasmid DNA in the presence or absence of topo I (alkylating and some genotoxic agents).

Salt Resistant Clamping of DNA to Tethered-Topo I

The presence of relaxed DNA products in the HTS was surprising since this DNA is covalently closed and circular and by definition, protein free. The retention of the relaxed circular DNA correlates with enzyme activity since heat inactivation of topo I significantly reduces DNA retention (FIG. 7A). The relaxed DNA is only partially released by high salt and 1% SDS was less effective. Since proteinase K completely eliminates this DNA, it is clearly dependent upon the physical presence of intact topo I. The fluorescent signal associated with relaxed DNA is also eliminated by DNase I digestion. It was concluded that relaxed, protein free DNA interacts with tethered topo I in the wells through unusually stable electrostatic or ionic bonding in a manner that differs from what is seen in free solution and represents salt (and detergent) resistant clamping of DNA. Some possible explanations are as follows (these are not mutually exclusive and a combination of effects cannot be ruled out).

i) Multiple protein-DNA contacts may exist concurrently. Crystal structures of non-covalent topo I/DNA complexes define topo I as a DNA clamp that surrounds B-form DNA. (Champoux J J: DNA TOPOISOMERASES: Structure, Function, and Mechanism. Annual Review of Biochemistry 2001 70:369). The HTS screens are performed with relatively high levels of topo I (molar ratios of enzyme:DNA about 100); therefore, a large number of clamping events are predicted per DNA molecule. Individual weak clamping structures may become much more stable in combination and resist ionic driven release. Even catalytically inactive mutant of topo I is able to efficiently capture plasmid DNA, and since this mutant can clamp, but not cleave DNA, the salt stable complexes may simply represent multiple protein/DNA contacts arising from the high stoichiometric excess of protein relative to DNA. Salt stable DNA clamping is also a hallmark of topoisomerase II; however, it is difficult to draw comparisons with topo I mechanisms. (Roca J: Topoisomerase II: a fitted mechanism for the chromatin landscape. Nucleic Acids Research 2007; 37:721-730; Roca J, Berger J M, Harrison C H, Wang J C: DNA transport by a type II topoisomerase: Direct evidence for a two-gate mechanism Proc Natl Acad Sci 1996; 93:4057-4062).

ii) Bound enzyme alters diffusion of the protein in solution resulting in complexes that cannot be easily washed free once bound. This prospect is related to (i) above but is more of a physical problem of trying to remove DNA from a surface. It is noted that the efficiency of the bound topo I in a hybrid assay (beads in solution, see FIG. 2) is much higher, which rules out the chemistry of the binding process as an explanation of the phenomenon.

iii) Topological structures form that are unique to the tethered enzyme. It cannot be ruled out that some unusual topological connectivity traps DNA on the plates. This possibility is supported by the fact that about 1% SDS did not reduce binding (FIG. 6A) which is a very effective protein denaturant. Thus, while unlikely, the formation of DNA nodes (DNA/DNA crossovers) may promote DNA interlocking structures, such as catenanes (a topo II mechanism). (Zechiedrich E, Osheroff, N.: Eukaryotic topoisomerases recognize nucleic acid topology by preferentially interacting with DNA crossovers. EMBO Journal 1990 9:4555-4562). While linears have not been detected, a necessary precursor structure, that does not mean fleeting and nested topo I cuts do not exist. Indeed, it was previously demonstrated that topo I tends to aggregate at sites of activity on DNA. (Mao Y M, I. Muller, M. T.: Nuclear and Nucleolar Localization by the N-terminal Domain of DNA Topoisomerase I. Proc Natl Acad Sci 2002 99:1235-1240). If topo I action is cooperative (where the first DNA/topo complex enhances the binding of the next one, and so forth) such topological structures could form. (SÃ, e K, Dianov G, Nasheuer H-P, Bohr V A, Grosse F, Stevnsner T: A human topoisomerase I cleavage complex is recognized by an additional human topisomerase I molecule in vitro. Nucleic Acids Research 2001 29:3195-3203). It is noted that the binding and titration isotherm (FIG. 5A) is sigmoidal rather than linear, which suggests cooperativity in binding.

Bound Topo I Displays Low Activity

One obvious concern with this solid phase assay is its inherently low efficiency. When tethered to a surface there is a loss of about 90% of the activity. There is no evidence for physical shearing or proteolytic degradation. Also, the low recovery is not something unique to His-tag Nickel binding chemistry, since low efficiency was not observed in a bead format. It seems more likely that a surface-fixed DNA binding protein cannot make productive contact with the template. Stated differently, protein scanning is impaired in a solid phase format. DNA binding proteins, like topo I, engage the template in a uni- or three dimensional search involving rapid exchanges of contacts over the surface of the DNA. In this way, the dimensionality of the search for a cleavage site is greatly reduced and allows any given ligand to find a specific DNA site at a rate that is actually faster than diffusion. (Hippel P H V, Revzin A, Gross C A, Wang A C: Non-specific DNA Binding of Genome Regulating Proteins as a Biological Control Mechanism: 1. The lac Operon: Equilibrium Aspects. Proceedings of the National Academy of Sciences of the United States of America 1974; 71:4808-4812; Roe J-H, Burgess R R, Record M T: Kinetics and mechanism of the interaction of Escherichia coli RNA polymerase with the [lambda]PR promoter. Journal of Molecular Biology 1984 176:495-522). When the ligand is sequestered to the surface, this scan/search process could be significantly hindered thereby reducing the efficiency of making productive contact with the substrate. In other words, DNA is diffusing freely through the entire volume of the well and since interaction with the surface bound ligand is through random collisions, the process is inherently inefficient.

Reconstruction HTS Assays with Known Compounds

The solid phase HTS method, summarized in FIG. 8, has value in detecting IFPs and CICs. The method is scalable with excellent Z′ values. Interpretation of the readout requires positive and negative controls (+/−CPT) and operates as a bimodal metric (HTS Ratios above about 1.0 would be candidate IFPs while ratios less than about 1.0 potential CICs).

Importantly, the method can potentially reveal drugs that specifically or non-specifically damage the ability of the enzyme to act, if such agents disrupt the DNA binding step (critical for enzyme action). In this case, the readout (fluorescence) would be reduced to some degree and the hit would be classified as a potential CIC (and may be a specific or non-specific inhibitor):

Any potential genotoxic agents that disrupt the DNA binding step can be detected with the method disclosed herein. The following examples are not meant to be construed in a limiting sense. Potential genotoxic agents that may be detected by the present invention include, for example, alkylating agents, DNA intercalating agents, estrogen modulators, topoisomerase inhibitors, etc.

It was observed that strong DNA intercalators (like mitoxantrone) strongly reduce DNA binding to the bound topo I. High concentrations of DNA intercalators are reported to inhibit topoisomerase activities; therefore, detection of a CIC is possible at least for DNA intercalators. (Wassermann K, Markovits J, Jaxel C, Capranico G, Kohn K W, Pommier Y: Effects of morpholinyl doxorubicins, doxorubicin, and actinomycin D on mammalian DNA topoisomerases I and II. Molecular Pharmacology 1990 38:38-45). With estrogen modulating drugs, Tamoxifen and Raloxifene, high ratios (over 2) were observed which was unexpected; however, these estrogen modulators have been reported to be topo I inhibitors. (Larosche I, LettÃ©ron P, Fromenty B, Vadrot N, Abbey-Toby A, Feldmann Gr, Pessayre D, Mansouri A: Tamoxifen Inhibits Topoisomerases, Depletes Mitochondrial DNA, and Triggers Steatosis in Mouse Liver. Journal of Pharmacology and Experimental Therapeutics 2007 321:526-535).

Moreover, Tamoxifen or an endogenous metabolite has been reported to be genotoxic (binding G residues and forming DNA adducts in vivo) and is a cationic drug. (Brown K: Is tamoxifen a genotoxic carcinogen in women? Mutagenesis 2009 24:391-404; Kim S Y, Suzuki N, Santosh Laxmi Y R, Shibutani S: Genotoxic Mechanism of Tamoxifen in Developing Endometrial Cancer. Drug Metabolism Reviews 2004 36:199-218). It is noted that Tamoxifen induced topo I cleavages (data not shown); however, the cleavages were less than with CPT, as expected.

Other genotoxic compounds, such as alkylating agents, are detected by HTS screening methods disclosed herein. It is rational to assume that such modifications in DNA affect either the non-covalent complex equilibrium (favor complex stability) or create a DNA suicide substrate (favors formation of cleavage complexes). While at first glance, one might view ‘off target’ hits as a problem, it is actually considered that this a potential bonus for the assay. Indeed, topo I might be an excellent probe for detecting genotoxic agents in general. This idea is based on finding that topo I cleavages can be readily detected in the absence of poisons (like CPT, FIG. 4).

In addition, it is noted that some of the alkylating agents that scored in the assay (Nitrogen mustard, Uracil mustard) induce single strand DNA nicks in the pHOT1 DNA in the absence of topo I (data not shown). In the HTS assays containing high topo I inputs, a DNA with a single strand nick would be more readily converted to a covalent complex (as a suicide substrate when topo I acts across from the nick). This may explain why some bifunctional alkylators are picked up by the assay. In many cases, the test drug did not significantly impact the metric (ratio) and would not be scored as a positive. For example, the ratio of about 1.4 as a cutoff was used because this is the value for topo I poisons Dactinomycin and Irinotecan. Thus, about 5 compounds out of about 50 scored as potential topo I IFP at about 100 uM input of purified test drug (single asterisk, FIG. 10). A relatively high input of test drug concentrations to amplify false positives was; however, in a typical HTS experiment the concentrations would be much lower and would detect far fewer false leads. For example, at about 10 uM test screens the number of positive hits was reduced (about 2 out of about 50, data not shown). If the stringency is increased to an HTS ratio of about 1.55 (Topotecan) the number of positive hits is further reduced.

As noted above, this topo I HTS assay is capable of detecting genotoxic agents (which are potential anticancer drugs) that are off target. However, secondary HTS would readily establish concentration dependence of the hits (it is noteworthy that with CPT as a prototype IFP, one would still see a strong positive readout). It is believed that this is due the high relative inputs of topo I used. On the other hand, this may also be an advantage for screening since it facilitates IFP detections at very low concentrations. In any positives; drug dependence would be gauged using standard biochemical criteria (cleavage assays in gels, in vivo complex formation or ICE bioassays, etc.). (Subramanian D, Furbee C S, Muller M T: ICE Bioassay. DNA Topoisomerase Protocols: Volume II: Enzymology and Drugs, 2000: 137-147; Trask D, DiDonato J, Muller M: Rapid Detection and isolation of covalent DNA/protein complexes: application to topoisomeraes I and II. EMBO Journal 1984 3:671-676; Trask D K, Muller M T: Stabilization of type I topoisomerase-DNA covalent complexes by actinomycin D. Proceedings of the National Academy of Sciences of the United States of America 1988 85:1417-1421).

Detecting catalytic inhibitors (CICs) using this method will most likely identify agents that disrupt topo/DNA scanning operations of the reaction cycle. Since this is a necessary antecedent step in the reaction toward breakage/rejoining, such drugs would be of immediate interest.

Unlike gel-based assays, which can quickly saturate at a stoichiometric maximum (see 4), the solid phase assay with picogreen operates over a wide range of proportionality (FIG. 5A). Therefore, by manipulating topo I loading of the wells, one could set up assays that are designed to identify CICs (low input) while high inputs would identify both IFCs and CIC.

To summarize the method described herein, His-tag purified topo I is bound to nickel coated plates in a standard binding buffer (typically about 200-500 units). Binding is complete within about 1 hr at room temperature and the reaction is optimized for 96 well plates in a about 100 uL volume. Free enzyme is washed out and pHOT1 supercoiled DNA is added in a topo I assay buffer. After incubating at about 37° C. for about 30 min, all DNA is about 100% relaxed and a fraction is bound to topo I in wells. Free DNA is then washed out and the relative fluorescence is measured using Picogreen staining. In the absence of CPT, the retained DNA is relaxed with some nicked open circular DNA (form II). In the presence of CPT, the retained DNA is mostly form II with a smaller amount of relaxed, protein free DNA. Relative fluorescence (RFU) is shown for a typical reaction with −/+controls (about 2000 and about 4000 fluorescent units respectively). The results are expressed as an “HTS Ratio” corresponding to the RFU of experimental to RFU of negative controls (no CPT). Based on reconstruction experiments, the experimental unknown will yield an HTS Ratio between zero and the positive CPT control. Thus, an IFP will result in an HTS Ratio greater than about 1.4-1.55 based on collective controls with other known topo I IFPs (FIG. 10); however, values greater than unity would be potential positives. The stringency for gating positives can be relaxed (about 1.4, Dactinomycin or Irinotecan, FIG. 10) or enhanced (Topotecan, 1.55) depending on total number of positives in any given screen. HTS Ratios near about 1.0 would not be scored as topo I effectors (FIG. 7B) and values <1.0 would be potential CICs (double asterisk, FIG. 10). The magnitude of the shift in HTS Ratio away from unity correlates with the potential intensity of topo I targeting. The HTS detects potential effectors which may be specific or non-specific topo I targeting agents. The former class of agents would be validated using conventional mechanistic testing (cleavage assays, ICE etc.). The latter non-specific agents might act on DNA and emulate either an IFP or a CIC. In the case of an IFP emulator, certain agents might nick the plasmid DNA substrate, or alter DNA structure (which increases the likelihood of a suicide event or trapping topo I/DNA complexes). In the case of a CIC emulator, the DNA may be stretched by an intercalator, making it unfit for engaging topo I or it may be bound by cationic drugs that inhibit formation of the electrostatic DNA clamp associated with non-covalent topo I binding.

The disclosures of all publications cited above are expressly incorporated herein by reference, each in its entirety, to the same extent as if each were incorporated by reference individually.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

It is also to be understood that the following claims are intended to cover all of the generic and specific features of the invention herein described, and all statements of the scope of the invention which, as a matter of language, might be said to fall there between. Now that the invention has been described,

What is claimed is:

1. A method of detecting agents having topoisomerase modulating activity comprising:
attaching a topoisomerase to a solid surface such that said topoisomerase retains activity;
contacting said topoisomerase with a polynucleotide having a sequence recognized by said topoisomerase; in the presence or absence of test agent suspected of modulating activity of said topoisomerase; and determining interaction of the topoisomerase with the polynucleotide by the test agent, wherein said determining comprises detecting an increase or decrease in association of the polynucleotide (or fragments thereof) with said topoisomerase.

2. The method of claim 1, wherein said polynucleotide is a DNA strand.

3. The method of claim 1, wherein said attaching comprises conjugating the topoisomerase with a tag component interactive with said solid surface.

4. The method of claim 3, wherein said tag is a His tag.

5. The method of claim 3, wherein said solid surface is coated with a metal.

6. The method of claim 5, wherein said metal is nickel.

7. The method of claim 1, wherein said determining step further comprises associating said polynucleotide contacted with said topoisomerase in the presence or absence of said test agent with a marker compound, and wherein an increase or decrease of marker associated with said polynucleotide is indicative of a change in modulation of said topoisomerase.

8. The method of claim 7, wherein said marker compound is a fluorescent dye.

9. The method of claim 8, further comprising comparing fluorescence of the fluorescent dye to a control signal, where the control signal is obtained by contacting only the topoisomerase, the DNA and the fluorescent dye.

10. The method of claim 9, wherein an increase in fluorescence indicates a potential interfacial poison and wherein a decrease in fluorescence indicates a potential catalytic inhibitor

11. A method of detecting topoisomerase modulating agents comprising:
conjugating a topoisomerase with a His-tag;
binding the topoisomerase to a solid surface;
contacting the topoisomerase with a DNA;
contacting the DNA with a fluorescent dye;
introducing an agent suspected of modulating activity of the topoisomerase;
comparing fluorescence of the fluorescent dye to a control signal, where the control signal is obtained by contacting only the topoisomerase, the DNA and the fluorescent dye; wherein an increase in fluorescence indicates a potential interfacial poison and wherein a decrease in fluorescence indicates a potential catalytic inhibitor.

12. The method of claim 11 wherein the solid surface is coated with a metal.

13. The method of claim 12 wherein the His-tag binds the topoisomerase to the solid surface.

14. The method of claim 11 wherein the fluorescent dye is picogreen.

15. An apparatus for detecting agents having topoisomerase modulating activity, the system comprising a solid support comprising topoisomerase bound to a surface thereof, wherein said topoisomerase retains activity.

16. The apparatus of claim 15, wherein said solid support comprises a microtiter plate.

17. The apparatus of claim 16, wherein said microtiter plate comprises a plurality of wells coated with metal.

18. The apparatus of claim 17, wherein said metal is nickel or cobalt.

19. The apparatus of claim 15, wherein said topoisomerase is bound to said surface via a His tag.

20. The apparatus of claim 15, wherein said solid support comprises a bead.

21. (canceled)

22. (canceled)

23. (canceled)

24. (canceled)

25. (canceled)

26. (canceled)

27. (canceled)

28. (canceled)

29. (canceled)
